(19) United States
(12) Patent Application Publication
(10) Pub. No.: US 2010/0255596 A1
(43) Pub. Date: Oct. 7, 2010

(54) CITRATE ANALYSIS FOR ELECTRODEPOSITION METHODS

(75) Inventors: NAZILA DADVAND, NEWTONVILLE, MA (US); BRENDAN THOR KEENE, NORTHBOROUGH, MA (US)

Correspondence Address: WOLF GREENFIELD & SACKS, P.C., 600 ATLANTIC AVENUE, BOSTON, MA, 02210-2206, US

(73) Assignee: Xtalic Corporation, Marlborough, MA (US)

(21) Appl. No.: 12/417,888

(22) Filed: Apr. 3, 2009

(51) Int. Cl. G01N 33/00

(52) U.S. Cl. 436/108; 436/128; 436/129

(57) ABSTRACT

Citrate analysis methods are described. In some embodiments, the methods can be used to analyze citrate in an electrodeposition bath.

### FIELD OF INVENTION

This invention relates generally to electrodeposition and, more particularly, to citrate analysis methods used in connection with electrodeposition methods and baths.

### BACKGROUND OF INVENTION

Electrodeposition is a common technique for depositing material on a substrate. Electrodeposition generally involves applying a voltage to a substrate placed in an electrodeposition bath to reduce metal ionic species within the bath which deposit on the substrate in the form of a metal, or metal alloy, coating. The voltage may be applied between an anode and a cathode using a power supply. At least one of the anode or cathode may serve as the substrate to be coated. In some electrodeposition processes, the voltage may be applied as a complex waveform such as in pulse plating, alternating current plating, or reverse-pulse plating.

A variety of metal and metal alloy coatings may be deposited using electrodeposition. For example, metal alloy coatings can be based on two or more transition metals. Tungsten-based coatings are one example of an electrodeposited coating. Such coatings may be tungsten alloys including one or more of the elements Ni, Fe, Co, B, S and P. These coatings often exhibit desirable properties, including high hardness, abrasion resistance, good luster, wear properties, coefficient of friction in sliding applications, amongst others.

Generally, the electrodeposition baths include one or more metal sources as well as additives that may improve the deposition process and/or the resulting coating. The metal source(s) may be selected based on the desired composition of the metallic coating on the article. Typical additives include complexing agent(s), wetting agent(s), brightening agent(s), leveling agent(s), carrier(s), ductility agent(s), and others.

Complexing agents may be depleted over time and/or during use; thus, there is an ongoing need for development of methods for analyzing complexing agents in an electrodeposition bath so that the amount of complexing agent in the bath can be adjusted to the proper level. In particular, there is a need for new methods for analyzing the complexing agent citrate in an electrodeposition bath that are compatible with the components in an electrodeposition bath and avoid separation steps.

### SUMMARY OF INVENTION

Citrate analysis methods are described.

In one aspect, a method is provided. The method comprises removing a sample from an electrodeposition bath comprising a tungsten and/or molybdenum ionic species, an ionic species of a second metal, and citrate. The method further comprises reacting the citrate and/or a citrate reaction product with a chemical reagent to form a product, reacting the product with a binding agent to form a complex, and analyzing the complex to determine the amount of citrate in the sample.

In another aspect, a method is provided. The method comprises removing a sample from an electrodeposition bath comprising a tungsten and/or molybdenum ionic species, an ionic species of a second metal, and citrate. The method further comprises analyzing a citrate reaction product using a spectroscopic technique to determine the amount of citrate in the sample.

In another aspect, a method is provided. The method comprises removing a sample from an electrodeposition bath comprising a tungsten and/or molybdenum ionic species, an ionic species of a second metal, and citrate and/or a citrate reaction product. The method further comprises diluting the citrate and/or citrate reaction product with a diluent, at least partially separating the citrate and/or citrate reaction product using HPLC, and analyzing the citrate and/or citrate reaction product by mass spectrometry to determine the amount of citrate in the sample.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### BRIEF DESCRIPTION OF DRAWINGS

FIG. 1 shows an electrodeposition system according to an embodiment.

FIG. 2 shows a citrate analysis flow chart according to an embodiment.

FIG. 3 shows a citrate analysis flow chart according to an embodiment.

### DETAILED DESCRIPTION

Citrate analysis methods are described. In some embodiments, the methods can be used to analyze citrate in an electrodeposition bath. Electrodeposition is a common technique for depositing material on a substrate. Electrodeposition generally involves applying a voltage to an article placed in an electrodeposition bath, which contains a mixture of chemicals including metal ionic species and additives. Applying the voltage reduces metal ionic species within the bath, which deposit on the substrate in the form of a metal, or metal alloy (e.g. nickel-tungsten alloy), coating. Citrate is an example of one type of additive that can be used as a complexing agent in an electrodeposition bath, which may keep metal species in the electrodeposition bath dissolved in the liquid. The methods disclosed herein provide techniques for analyzing citrate that involve transforming the citrate and/or a citrate reaction product into a new species that can form a complex in a subsequent step. The complex may be analyzed to determine the amount of citrate in the electrodeposition bath. The amount of citrate in the electrodeposition bath can then be adjusted to a desired level, which is important for keeping metal species dissolved in the electrodeposition bath.

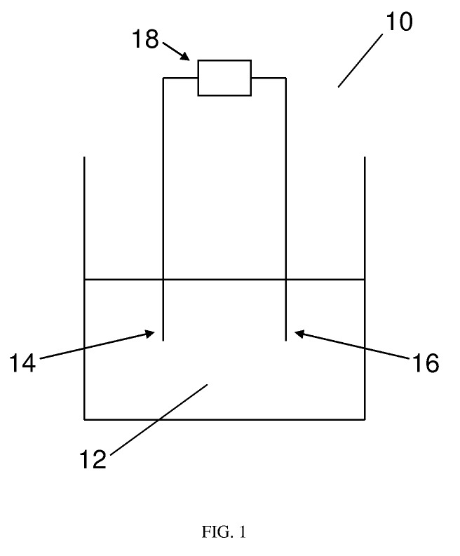

FIG. 1 shows an electrodeposition system 10. The system includes a electrodeposition bath 12. As described further below, the bath includes the metal sources (e.g. nickel-tungsten sources) used to form the coating and one or more additives, which may include a citrate as a complexing agent. An anode 14 and cathode 16 are provided in the bath. A power supply 18 is connected to the anode and the cathode. During use, the power supply generates a waveform which creates a voltage difference between the anode and cathode. The voltage difference leads to reduction of metal ionic species in the bath which deposit in the form of a coating on the cathode, in this embodiment, which also functions as the substrate.

It should be understood that the illustrated system is not intended to be limiting and may include a variety of modifications as known to those of skill in the art.

The electrodeposition baths comprise a fluid carrier for the metal source(s) and additive(s). In some embodiments, the fluid carrier is water. However, it should be understood that other fluid carriers may also be used such as molten salts, cryogenic solvents, alcohol baths, amongst others. Those of ordinary skill in the art are able to select suitable fluid carriers.

Generally, the pH of the electrodeposition bath can be from about 2.0 to 12.0. In some cases, the electrodeposition bath may have a pH from about 7.0 to 9.0, or, in some cases, from about 7.6 to 8.4, or, in some cases, from about 7.9 to 8.1. However, it should be understood that the pH may be outside the above-noted ranges.

In some cases, the operating range for the electrodeposition baths described herein is 30-100° C., 40-90° C., 50-80° C., or, in some cases, 50-70° C. However, it should be understood that other temperature ranges may also be suitable.

The baths include suitable metal sources for depositing a coating with the desired composition. When depositing a metal alloy, it should be understood that all of the metal constituents in the alloy have sources in the bath. The metal sources are generally ionic species that are dissolved in the fluid carrier. As described further below, during the electrodeposition process, the ionic species are deposited in the form of a metal, or metal alloy, to form the coating. In general, any suitable ionic species can be used. The ionic species may be metal salts. For example, sodium tungstate, ammonium tungstate, tungstic acid, etc. may be used as the tungsten source when depositing a coating comprising tungsten; and, nickel sulfate, nickel hydroxy carbonate, nickel carbonate, nickel hydroxide, etc. may be used as the nickel source to deposit a coating comprising tungsten. It should be understood that these ionic species are provided as examples and that many other sources are possible.

As described herein, the electrodeposition baths may include one or more components (e.g., additives) that may enhance the performance of the baths in producing coated articles. In some embodiments, the electrodeposition bath may comprise one or more complexing agents, brightening agents, wetting agents, etc. Suitable bath compositions have been described in U.S. patent application Ser. No. 12/266,979, filed Nov. 7, 2008, which is incorporated herein by reference in its entirety.

As noted above, citrate may be used as an additive, which functions as a complexing agent. A complexing agent is a known term in the art and generally refers to a species that can associate (i.e., coordinate, chelate, etc.) with the metallic ions contained in the bath. The complexing agent may be an organic species, such as citrate. In some cases, other complexing agents, such as inorganic species (e.g. an ammonium ion), neutral species, or other charged species (e.g., negatively charged ion, positively charged ion) may be used in addition to citrate. Examples of other complexing agents include gluconates, tartrates, and other alkyl hydroxyl carboxylic acids.

As used herein, the term “citrate” includes the compound citrate itself as well as substituted derivatives of citrate. For example, citrate, or substituted derivatives thereof, may have the following structure,

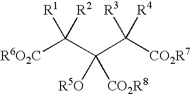

wherein each R¹-R⁴ may be the same or different and is hydrogen, halogen, alkyl, alkenyl, aryl, acyl, nitro, cyano, hydroxy, or the like, and R⁵-R⁸ may be hydrogen, halogen, alkyl, alkenyl, aryl, acyl, or a pair of electrons (i.e., a carboxylate). In some embodiments, each R¹-R⁵ is hydrogen and each R⁶-R⁸ is hydrogen (e.g., citric acid), a pair of electrons (i.e., a deprotonated and negatively charged carboxylate group) (e.g., citrate), or a mixture of hydrogen and electrons (e.g., hydrogen citrate and/or dihydogen citrate). R¹-R⁸ may be selected such that the citrate compound, or substituted derivative thereof, does not substantially interfere with the electroplating process. For example, R¹-R⁵ may be selected such that the ability of the citrate compound to chelate a metal ion is not substantially affected, or, in some cases, is improved. In some cases, at least one of R⁶-R⁸, at least two of R⁶-R⁸, or each of R⁶-R⁸ is a capable of forming a negative charge. Citrate may also be modified in preparation for analysis, described in detail below. Those of ordinary skill in the art would be able to select appropriate citrate derivatives for use in the invention.

Citrate may react to form reaction products. In some embodiments, the citrate in an electroplating bath reacts to form at least one reaction product that may be useful for determining the amount of citrate in the bath. For example, citrate may react to form aconitate, or a substituted derivative thereof, having the following structure,

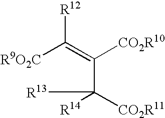

wherein R⁹-R¹⁴ may be the same or different and is hydrogen, halogen, alkyl, alkenyl, aryl, acyl, nitro, cyano, hydroxy, or the like, and R⁹-R¹¹ may be hydrogen, halogen, alkyl, alkenyl, aryl, acyl, or a pair of electrons (i.e., a carboxylate). In some embodiments, each R¹²-R¹⁴ is hydrogen and R⁹-R¹¹ are hydrogen (e.g., aconitic acid), a pair of electrons (i.e., a deprotonated and negatively charged carboxylate group) (e.g., aconitate), or a mixture of hydrogen and electrons (e.g., hydrogen aconitate and/or dihydogen aconitate). R⁹-R¹⁴ may be the same or different from the corresponding groups on the parent citrate molecule. Additionally, aconitate may exist in a cis conformation instead of or in addition to the trans conformation shown above.

In some embodiments, citrate may react to form oxalate, or a substituted derivative thereof, having the following structure,

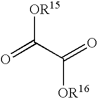

wherein R¹⁵ and R¹⁶ may be the same or different and are hydrogen, halogen, alkyl, alkenyl, aryl, acyl, or a pair of electrons (i.e., a carboxylate). In some embodiments, R¹⁵ and R¹⁶ are hydrogen (e.g., oxalic acid), a pair of electrons (i.e., a deprotonated and negatively charged carboxylate group) (e.g., oxalate), or a mixture of hydrogen and electrons (e.g., hydrogen oxalate). R¹⁵ and R¹⁶ may be the same or different from the corresponding groups on the parent citrate molecule.

Citrate may also react to form malonate, or a substituted derivative thereof, having the following structure,

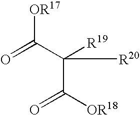

wherein R¹⁹ and R²⁰ may be the same or different and are hydrogen, halogen, alkyl, alkenyl, aryl, acyl, nitro, cyano, hydroxy, or the like, and R¹⁷ and R¹⁸ may be hydrogen, halogen, alkyl, alkenyl, aryl, acyl, or a pair of electrons (i.e., a carboxylate). In some embodiments, R¹⁹ and R²⁰ are hydrogen and R¹⁷ and R¹⁸ are hydrogen (e.g., malonic acid), a pair of electrons (i.e., a deprotonated and negatively charged carboxylate group) (e.g., malonate), or a mixture of hydrogen and electrons (e.g., hydrogen malonate). R¹⁷-R²⁰ may be the same or different from the corresponding groups on the parent citrate molecule.

In some instances, citrate may react to form glutarate, or a substituted derivative thereof, having the following structure,

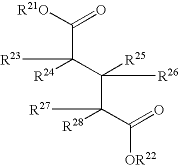

wherein R²³-R²⁸ may be the same or different and is hydrogen, halogen, alkyl, alkenyl, aryl, acyl, nitro, cyano, hydroxy, or the like, and R²¹ and R²² may be hydrogen, halogen, alkyl, alkenyl, aryl, acyl, or a pair of electrons (i.e., a carboxylate). In some embodiments, each R²³-R²⁸ is hydrogen and R²¹ and R²² are hydrogen (e.g., glutaric acid), a pair of electrons (i.e., a deprotonated and negatively charged carboxylate group) (e.g., glutarate), or a mixture of hydrogen and electrons (e.g., hydrogen glutarate). R²¹-R²⁸ may be the same or different from the corresponding groups on the parent citrate molecule.

Citrate may also react to form a variety of other reactions products including, but not limited to, maleate, fumarate, succinate, tricarballylate, lactate, malate, glycolate, tartarate, and citraconate. As described above, the citrate reaction products may be substituted with a variety of functional groups on carbon and/or oxygen atoms within the reaction products. The substitutions may be the same or different from the corresponding groups on the parent citrate molecule.

In some embodiments, citrate may react to form a plurality of reaction products (i.e., a mixture of reaction products). Furthermore, the relative amounts of each reaction product formed may be essentially constant for a particular condition (i.e., a particular electrodeposition bath condition). For example, under one electrodeposition bath condition, aconitate may constitute a certain proportion of the reaction product mixture (e.g., about 80%), whereas under another electrodeposition bath condition, aconitate may constitute a different proportion of the reaction product mixture.

Without wishing to be bound by any theory, the principle of conservation of mass reveals that the initial mass of citrate is equal to the mass of any citrate reaction products that form from this initial mass of citrate plus the mass of the remaining amount of the initial mass of citrate. Accordingly, for an electrodeposition bath where at least some of the citrate reacts over time to form at least one reaction product, the amount of citrate remaining in the bath can be calculated by analyzing the citrate in the electrodeposition bath and/or analyzing at least one citrate reaction product. For example, the concentration of citrate in an electrodeposition bath can be derived from the concentration of a citrate reaction product in the electrodeposition bath using the formula Cₜ=C₀−(Rₜ/P), where C is the molar concentration of citrate at time t, C₀ is the initial molar concentration of citrate, Rₜ is the molar concentration of citrate reaction product at time t, n is the stoichiometry of the reaction, and P is the molar fraction of the total citrate reaction product mixture that corresponds to reaction product R. It is understood that this equation is general and that Rₜ could be a summation of the concentration of at least two reaction products and P could be the corresponding fraction of the reaction product mixture that the at least two reaction products occupy.

Generally, a complexing agent, or mixture of complexing agents, may be included in the electrodeposition bath within a concentration range of 10-200 g/L, and, in some cases, within the range of 40-80 g/L. In some embodiments, ammonium ions may be incorporated into the citrate-containing electrodeposition bath as complexing agents and to adjust solution pH. For example, the electrodeposition bath may comprise ammonium ions in the range of 1-50 g/L, and between 10-30 g/L.

In some aspects, various techniques can be used to monitor the contents of the electrodeposition baths. For example, the techniques may determine the concentration of citrate in a bath containing other additives such as brightening agent(s), wetting agent(s), complexing agent(s), etc. If the concentration of the citrate is below or above a desired concentration, the bath composition may be adjusted so that the concentration lies within the desired range.

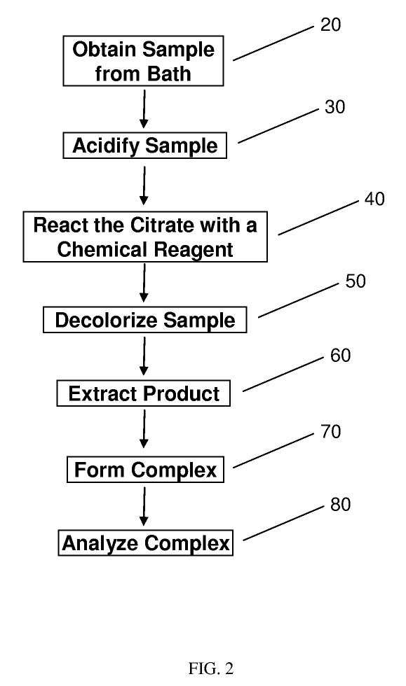

FIG. 2 provides a flow chart depicting the various steps of citrate analysis according to one embodiment of the invention. In some embodiments, the analysis begins with step 20 which involves obtaining a sample from an electrodeposition bath. In some cases, the sample may be acidified as in step 30, for example to prepare the sample for step 40, the reaction of a citrate and/or citrate reaction product with a chemical reagent to form a product. In some embodiments, the chemical reagent may interfere with the analysis method, so the sample may be decolorized in step 50 after reacting the citrate and/or citrate reaction product with a chemical reagent in step 40. The next steps may involve extracting the product from the sample in step 60 and forming a complex between the product and a binding agent in step 70. The complex may then be analyzed in step 80 using techniques as described below. It should be understood that some methods may not include all of these steps. For example, in some cases the sample may have a pH value that obviates the acidification step 30. In other instances, the sample may not be decolorized and/or treated to quench the chemical reagent. In some examples, the extraction step 60 may be excluded.

In some embodiments, a sample may be taken from an electrodeposition bath for analysis as in step 20. The sample may be taken at various intervals, for example once per hour, once per day, etc. It should be understood that other intervals could also be used. In some instances, the sample may be taken prior to and/or following the electrodeposition of an article. In some cases, the change in citrate level may be followed over a period of time by sampling the electrodeposition bath at a plurality of time intervals. For example, monitoring the citrate level as a function of time may be useful for estimating the time point at which the citrate level will fall below a desired level.

In some embodiments, the sample may be diluted. For example, the sample may be diluted prior to analyzing the citrate and/or citrate reaction product. The sample may also be diluted during analysis of the citrate and/or citrate reaction product. In some cases, diluting the sample can improve the accuracy of the methods for analyzing the citrate and/or citrate reaction product. For example, a liquid (e.g. water) may be added to the sample to produce about a 1:1,000 dilution. It is understood that other dilution factors also may be useful. In some instances, the sample may contain an amount of citrate and/or citrate reaction product for which diluting the sample would not result in an improvement in citrate and/or citrate reaction product analysis accuracy. The initial volume of the sample taken from the bath may be about 0.1 mL, although other volumes are also suitable.

In some methods, the pH of a sample is adjusted as in the acidification step 30. Generally, the pH adjustment prepares the sample for the reaction in step 40. For instance, in some cases, the reaction rate in step 40 increases as the pH of the sample decreases. In one example, the pH may be adjusted to less than about pH 0. In other instances, the pH may be adjusted to a value less than about 1, less than about 2, less than about 3, etc. In some cases, acid is added to the sample. In one example, a solution of metaphosphoric acid and sulfuric acid is added to the sample. Those skilled in the art will recognize that other acids and/or acid combinations are suitable for addition to the sample instead of or in addition to metaphosphoric acid and sulfuric acid. Generally, the addition of an acidic solution to a less acidic solution causes energy, for example heat, to be released. Thus, in some embodiments, the sample is cooled after addition of acid to the sample. In other embodiments, the sample is cooled prior to addition of acid to the sample. In some instances, the acidified sample is adjusted to room temperature prior to performing a subsequent step in a citrate analysis method.

In some embodiments, having obtained a sample and, in some cases, adjusted the pH, the citrate and/or citrate reaction product in the sample may be reacted to form a new species (i.e., a product) as in step 40. Generally, the purpose of reacting the citrate and/or citrate reaction product is to form a new species that may be a better analysis substrate. In some embodiments, chemical reagents that react with citrate and/or citrate reaction product to break and/or form covalent bonds may be used. For example, an oxidizing agent and a halogen may be reacted with citrate and/or citrate reaction product. Combining these reagents with citrate and/or citrate reaction product can, in some cases, result in the formation of a product. In some instances the product contains a halogen. As described below, a product containing a halogen may form a complex that can be analyzed. Oxidizing agents include, but are not limited to, permanganate species (e.g. potassium permanganate, etc.), chromate species (e.g. potassium chromate, potassium dichromate, etc.), etc. Various halogen species may be used according to some embodiments of the invention, including bromide species (e.g. sodium bromide, potassium bromide, etc.), chloride species (e.g. sodium chloride), fluoride species (e.g. sodium fluoride), and/or iodine species (e.g. sodium iodide). Reaction of citrate and/or citrate reaction product with a halogen may result in the formation of a halogenated product, i.e. pentabromoacetone.

In some instances, the oxidizing agent and halogen species are added to the sample together. The rate of addition of the oxidizing agent and halogen species to the sample may be controlled such that the addition occurs over a period of time that maximizes the yield of the reaction, for instance about 20 minutes. It should be recognized that other times are also suitable.

In some instances, conducting the reaction at a low temperature can maximize the yield of the product. The reaction, in some cases, may be conducted at a temperature between about −10° C. and 10° C., between about −5° C and 5° C., between about 0° C. and 3° C., etc.

In some embodiments, the amount of chemical agents (e.g. oxidizing agent and halogen species) added to the sample stoichiometrically exceeds the amount of citrate and/or citrate reaction product in the sample. Those skilled in the art would be able to calculate the amount of oxidizing agent and halogen species to add by, for example, calculating the amount of citrate that would be present in the sample assuming the amount of citrate did not change during the time between addition of citrate to the electrodeposition bath and withdrawal of the sample from the electrodeposition bath (i.e. calculating the maximum expected amount of citrate in the sample) and adding an amount of oxidizing agent and halogen species that stoichiometrically exceeds the calculated amount of citrate.

Step 50 refers, in some embodiments, to treating the sample in a way that decreases interference with the analysis methods. In some instances, decolorization of the sample may be carried out following reaction of the citrate and/or citrate reaction product to form a new species. For example, a hydrogen peroxide solution may be added until the sample becomes colorless. In another example, an organic peroxide may be used. Those skilled in the art will be able to select other agents for decolorizing the sample. Without wishing to be bound by any theory, the decolorization occurs by destroying unreacted oxidizing agent. Thus, other agents that destroy unreacted oxidizing agent would be recognized by those skilled in the art as having utility in the present invention. In some cases, other reagents that interfere with the analysis methods can be destroyed using methods known to those skilled in the art.

Step 60 refers to extraction of the product from the sample. In certain embodiments, the product can be extracted from the sample using an organic solvent. For example, organic solvents that form a partition with water and in which the product is soluble may be suitable for the extraction. One example of a suitable organic solvent is heptane. Some examples of alternative organic solvents are hydrocarbons, such as pentane, hexane, octane, nonane, isomers thereof, etc.

In some embodiments, the product is extracted from the aqueous sample by agitating the aqueous and organic solvent mixture. For instance, the aqueous and organic solvent mixture may be shaken together for a period of time to extract the product. In some examples, the mixture can be agitated for about 10 minutes. It is understood that other times are also suitable. In other embodiments, the agitation may be performed manually and/or mechanically.

The vessel in which the aqueous and organic solvent mixture are contained may allow residual chemicals to remain, for example on the walls of the vessel. In some cases, residual chemicals can interfere with the analysis methods, so it may be advantageous to remove these chemicals prior to analysis. In some instances, residual chemicals, for example acids, may be removed by transferring the aqueous phase from a first vessel to a second vessel. In some examples, the first vessel may be cleaned before returning the aqueous phase to it. In other examples, the aqueous phase may be extracted more than once. For instance, the aqueous phase may be extracted with an organic solvent in a first vessel, the organic solvent may be removed to a second vessel and the aqueous phase to a third vessel. The aqueous phase may then be extracted a second time. Those skilled in the art will recognize that this process may be repeated and that any of the vessels may be cleaned and reused. In some embodiments, the aqueous phase of an aqueous and organic solvent mixture in a vessel may be discarded after being extracted. Water may be added to the vessel, the vessel may be agitated to wash the vessel, and the water may be discarded. This process may be repeated more than once.

In some embodiments, the product may form a complex as in step 70. Generally, the complex is more readily analyzed than the product. In some instances, the complex may be formed by adding a binding agent to a solution containing the product. For example, an organic solvent may contain the product, and an aqueous solution containing the binding agent may be added to the organic solvent. In some instances, the aqueous solution may contain a buffer in addition to the binding agent. One example of a binding agent is thiourea. Various buffers, such as sodium borate, are known to those skilled in the art and may be used. In some instances, the formation of a complex may be pH sensitive and may benefit from the pH stabilization that a buffer can provide. In some embodiments the pH of the binding agent solution may be about 9.2.

In some examples, the complex may be formed by agitating the aqueous solution and the organic solution. For instance, the two solutions may be mixed for about 5 minutes using a mechanical shaker. In some embodiments, the solutions may be agitated for less than about 5 minutes or greater than about 5 minutes. Complexes comprising thiourea and a brominated product may be soluble in aqueous solutions and may migrate to the aqueous portion of an aqueous and organic solvent mixture. The aqueous layer may be separated from the organic solvent for subsequent analysis.

Step 80 refers to analysis of the complex. In some embodiments, complexes of thiourea and a brominated product may be analyzed. Certain complexes may have a yellow color that is proportional to the amount of complex in a solution. In some embodiments, the aqueous portion of an aqueous and organic solvent mixture may be isolated. Analysis of the complex may be achieved using a variety of techniques known in the art, for example spectrophotometry, colorimetry, etc. In some embodiments, the complex may be analyzed at 448 nm. The absorbance of the complex at 448 nm is proportional to the concentration of the complex in the solution and may be used to calculate the concentration of citrate in the sample. It is understood that the solution containing the complex may be made more concentrated or diluted and still be useful for analyzing the complex. A standard curve for citrate concentration versus absorbance can be generated, for example, using the absorbance values of a set of solutions that contained a range of known concentrations of citrate and/or citrate reaction product and plotting the absorbance values against the known concentrations of citrate and/or citrate reaction product. For each solution containing a known concentration of citrate and/or citrate reaction product, the methods as described above are performed to generate the complex, which is then analyzed. Fitting a curve to the resultant plot, using a method such as a linear regression, can allow the derivation of a general mathematical formula for calculating the concentration of citrate in a sample having an unknown concentration of citrate by inputting the absorbance value of the sample, after being reacted to form the complex, into the formula.

In another embodiment, the extinction coefficient of the complex at a particular wavelength may be calculated and used to determine the concentration of complex in solution using techniques known to those skilled in the art. For example, a series of solutions with each solution having a different concentration of the complex may be prepared. The absorbance of each solution can be measured, for example at 448 nm, and the absorbance of each solution can be plotted as a function of the concentration of the complex. The slope of the resultant curve is the extinction coefficient for the complex at the wavelength used to measure the absorbance of the solutions. The concentration of the complex in a sample having an unknown concentration of complex can be determined using the extinction coefficient by measuring the absorbance of the sample at the wavelength that corresponds to the extinction coefficient as understood by those skilled in the art.

In some cases, stock solutions of reagents used in the above described methods may change over a period of time that can reduce the effectiveness of a reagent. Thus in some embodiments, stock solutions of reagents may be freshly prepared within the same 24 hour period during which the citrate analysis method will be performed. For example, oxidizing solution and/or the complex solution may be freshly prepared prior to use.

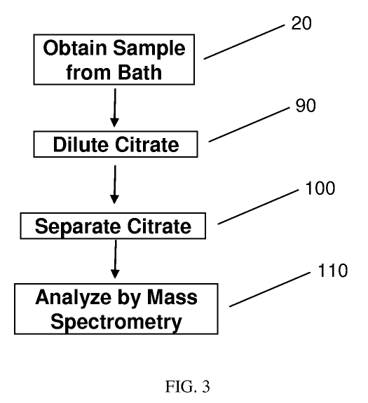

FIG. 3 provides a flow chart depicting the various steps of citrate analysis according to another embodiment of the invention. In some cases, the analysis begins with step 20, which involves obtaining a sample from an electrodeposition bath as described above. The sample may be diluted, in some instances, as in step 90, for example to prepare the sample for separation in step 100. The sample may then be analyzed by mass spectrometry in step 110 as described below.

In some embodiments, the sample may be diluted prior to separation as shown in step 90. The sample may also be diluted during separation or after separation. In cases where the sample is analyzed by mass spectrometry, the concentration of citrate and/or citrate reaction product may affect the accuracy and/or precision of the analysis. In some instances, the sample may be diluted such that the concentration of citrate and/or citrate reaction product is less than about 100 parts per million (ppm), less than about 50 ppm, less than about 30 ppm, etc. In other examples, the concentration of citrate and/or citrate reaction product may be between about 10 ppm and about 25 ppm.

The sample may be diluted with water and/or a diluent comprised of water, solutes, organic solvents, etc. In some instances, the sample may be diluted more than once (e.g., serially diluted).

In cases where the sample is separated using liquid chromatography (e.g., HPLC), the sample may be diluted with a diluent that is compatible with the HPLC method. For example, the polarity of the diluent may be different than the polarity of the mobile phase. “Mobile phase” refers to the fluid component of a chromatography system (i.e., the aqueous and/or organic solvents that flow through a chromatographic column. In some cases, the polarity of the diluent may be higher than the polarity of the mobile phase (i.e., the polarity of the diluent may be more polar than the polarity of the mobile phase). For example, the polarity of the diluent may be more polar than the initial polarity of the mobile phase.

The polarity of a solvent, such as the diluent or mobile phase, may be adjusted by adding one or more components to the solvent. For example, the polarity of an aqueous solvent may be adjusted by the addition of an organic solvent having a different polarity than the aqueous solvent. Generally, the increasing amount of organic solvent in an aqueous solution decreases the polarity of the aqueous solution. The organic solvent should typically be miscible with the mobile phase. For example, when the mobile phase is aqueous, the organic solvent should be miscible with water. Examples of miscible organic solvents include alcohols such as methanol, ethanol, and isopropanol, acetonitrile, acetone, etc. In some embodiments, an organic solvent may not be miscible in the mobile phase but may be soluble in the mobile phase up to a certain concentration. An example of a water soluble organic solvent is tetrahydrofuran.

The diluent may contain various additives that affect the eluting power of the diluent. In some embodiments the diluent contains a buffer. In certain instances, a volatile buffer may be used (i.e., a buffer that can evaporate or sublime). Volatile buffers typically comprise a volatile acid (e.g., formic acid, acetic acid, carbonic acid etc.) and a volatile base (e.g., amines such as ammonium hydroxide, triethylamine, etc.). Examples of volatile buffers include ammonium acetate, ammonium formate, triethylammonium acetate, triethylammonium formate, etc.

An acid and/or base may be added to the diluent to adjust the pH. In some examples, the acid and/or base may be volatile. For example, formic acid may be added to the diluent to adjust the pH lower. Similarly, ammonium hydroxide may be added to adjust the pH higher.

In one example, the diluent may be an aqueous solution comprised of about 2 mM ammonium acetate, about 30% v/v methanol, about 0.07% v/v formic acid, and about 0.1% v/v acetonitrile. Those skilled in the art will recognize that these concentrations may be adjusted and that the effectiveness of these adjustments can be readily tested by analyzing a sample using HPLC and comparing the effectiveness of the analysis under the test conditions to the analysis using the diluent described above. For example, the ammonium acetate concentration may be between about 0.2 mM and about 200 mM, the methanol concentration may be between about 10% and about 50%, the formic acid concentration may be less than about 1% and the acetonitrile concentration may be less than 10%, less than 50%, etc. In some embodiments, acetonitrile is not used. Acetonitrile may also be used in place of methanol.

As shown in step 100, the sample may be separated (i.e., at least one component of a mixture may be at least partially separated from at least one other component of a mixture). A variety of means may be used to at least partially separate at least one component of a mixture, for example liquid chromatography techniques such as HPLC. In some embodiments, reversed-phased HPLC can be used to separate the sample. A non-limiting example of a reversed-phase HPLC column is an Atlantis T3 column (sold by Waters Corporation, http://www.waters.com) having dimensions of 2.1 mm wide by 50 mm long and packed with a solid phase comprising C18 functionalized silica particles (3 micron particle size, 100 Angstrom pore size). Without wishing to be bound by any theory, in reversed-phase HPLC an analyte interacts with the solid phase through hydrophobic interactions. Organic solvents in the mobile phase that interfere with the hydrophobic interactions between an analyte and the solid phase generally cause faster elution of the analyte. Those skilled in the art will recognize that other columns and column parameters may also be used in the methods.

HPLC separation may include a binary solvent system. The polarity of the first solvent may be different than the polarity of the second solvent such that the polarity of the mobile phase can be adjusted by changing the proportion of the first solvent to the second solvent in the mobile phase. In some instances, the first solvent may have a weaker elution strength than the second solvent. For example, the first solvent may be an aqueous solution comprising a salt, such as ammonium acetate, and may include acidic additive, such as formic acid (e.g., about 2 mM ammonium acetate and about 0.1% v/v formic acid). The second solvent generally comprises an organic solvent (e.g., essentially 100% methanol). A simple test for comparing the elution strengths of two candidate solvents is to compare the elution time of an analyte using each candidate solvent under similar conditions. The candidate solvent that results in a longer elution time for the analyte has the weaker elution strength. In some embodiments, the eluting power of the diluent may be weaker than that of the mobile phase.

A mixture of the two solvents may be used to elute the sample from the column. In some cases, the elution may be isocratic (i.e., the mobile phase composition is constant). For example, the sample may be eluted using a mobile phase composition of 20% of the first solvent and 80% of the second solvent. In other cases, the elution may involve a gradient of the two solvents over a period of time. For example, an initial mobile phase composition of 90% of the first solvent and 10% of the second solvent may be adjusted to 30% of the first solvent and 70% of the second solvent over a period of time to elute the sample. In some instances, the initial mobile phase composition may be defined as the mobile phase composition when the sample is injected into the HPLC system. In other instances, the initial mobile phase composition may be defined as the mobile phase composition when the sample enters the HPLC column. In some cases, the gradient may be linear (i.e. changing at a constant rate). Some elution methods may involve both gradient periods and isocratic periods. The flow rate of the eluent may be fixed or variable.

In some embodiments, the sample may be analyzed by mass spectrometry as shown in step 110. The eluent from chromatographic separation may be fed into the mass spectrometer to allow analysis of the separated sample. In some examples, performing mass spectrometry on a sample after HPLC separation improves the peak shape on the mass spectrum. Integration of the peak area on a mass spectrum can allow quantification of an analyte.

In some cases, tandem mass spectrometers may be used to analyze a sample. For instance, a sample may be fed into a first mass spectrometer, and ions having a mass to charge ratio within a specific range can be selectively fed into a second mass spectrometer. In the second mass spectrometer, for example, the selected ions can be fragmented and analyzed. Without wishing to be bound by any theory, fragmentation of an ionized molecule generally produces a variety of ions of different mass, and within a sample, the mass to charge ratio of one or more of these ions can be unique. Consequently, analysis of an ion having a unique mass to charge ratio within a sample can allow quantification of an analyte without interference from contaminating ions having a similar mass.

In some embodiments, the volatility of the eluent can affect the performance of a mass spectrometer. For example, the inclusion of an organic solvent in an aqueous effluent can improve the vaporization of the effluent and the ionization of the analyte, leading to better analysis of the analyte. Conversely, in some cases the presence of an organic solvent in an effluent can decrease the quality of analysis (e.g., by decreasing the signal to noise ratio) if the concentration of organic solvent is too high. For example, the concentration of methanol in an aqueous effluent may be between about 10% and about 50%. Additionally, the amount of volatile salts, acid, and bases can also affect the performance of a mass spectrometer. Those skilled in the art will be able to adjust the concentrations of additives to achieve suitable performance from the mass spectrometer.

In some instances, analysis of a sample by a mass spectrometer produces a signal that can be quantified. For example, signal may have an intensity (i.e., signal height) and duration, which forms a mathematical curve (i.e., a peak). Integration of the area under this curve can yield a quantity (i.e., an integral) that is proportional to the amount of an analyte in the sample. In some embodiments, a standard curve (discussed above) may be constructed that correlates a known amount of citrate and/or citrate reaction product with an integral value. Fitting a curve to the resultant plot, using a method such as a linear regression, can allow the derivation of a general mathematical formula for calculating the concentration of citrate in a sample having an unknown concentration of citrate by inputting the integral value, obtained from analysis by mass spectrometry, into the formula.

In some cases, a spectroscopic technique can be used to analyze a sample. For example, the effluent from an HPLC containing an analyte can be flowed through a detector such as a UV-vis spectrophotometer. The signal produced by the detector in response to light absorption by the analyte can be recorded and used to quantify the amount of analyte in the sample. For example, the signal may have an intensity (i.e., signal height) and duration, which forms a mathematical curve (i.e., a peak). Integration of the area under this curve can yield a quantity (i.e., an integral) that is proportional to the amount of an analyte in the sample. The signal generated from absorption of one or more wavelengths of light can be used. In some embodiments, a sample can be analyzed by UV-vis spectrophotometry without the use of HPLC. For example, a sample can be taken from an electrodeposition bath and directly analyzed. A sample may also be diluted prior to analysis, for example to bring the concentration of the analyte in the sample to within a suitable range for detection.

In certain embodiments, citrate and/or citrate reaction product may have an extinction coefficient at a particular wavelength that allows the molecule to be analyzed by UV-vis spectrophotometry. Those skilled in the art will be able to select a suitable wavelength for analysis of citrate and/or citrate reaction product.

The analytical methods described herein address a fundamental problem in the field. In some cases, additives such as the citrate complexing agent should be present at a level that confers function to an electrodeposition bath. Maintaining the additive in the proper amount is thus important to the value of the additive in the electrodeposition bath. In the art, concentration is often used as the metric for the level of an additive in a bath and may be defined as the amount of additive per amount of bath. It is understood that “amount” can be in units of mass, weight, volume, moles, etc. Typically, concentration is given in units of mass per volume. However, other definitions of concentration may be used by those skilled in the art. For example, the concentration of additive may be defined as the amount of additive per amount of another additive.

The speed with which citrate can be analyzed using these methods allows analysis at multiple time points in a single day. Since the level of citrate in an electrodeposition bath can vary on a timescale of hours, the ability to assay citrate, and subsequently adjust the level of citrate if necessary, is important for maintaining the quality of electrodeposition.

As the baths can contain a plurality of additives, a challenge exists to analyze a specific additive in the presence of others. Due to the chemical structure, analysis of an additive in its native form may be insensitive and/or time consuming. Additionally, other additives may interfere with analysis of a target additive. The methods disclosed herein circumvent these issues. For example, the citrate analysis provides methods for analyzing citrate in an electrodeposition bath containing a complex mixture of chemicals, thereby allowing the methods to be used with many electrodeposition bath compositions. In some embodiments, the methods do not require separation of citrate and/or citrate reaction product (e.g. by chromatography) from the other components of the electrodeposition bath, which reduces the time and cost of analysis. As mentioned above, in some embodiments, the electrodeposition bath may include additional additives in addition to the citrate complexing agent. For example, the electrodeposition bath may include at least one brightening agent. The brightening agent may be any species that, when included in the baths described herein, improves the brightness and/or smoothness of the metal coating produced. In some cases, the brightening agent is a neutral species. In some cases, the brightening agent comprises a charged species (e.g., a positively charged ion, a negatively charged ion). In one set of embodiments, the brightening agent may comprise an alkyl group, optionally substituted. In some embodiments, the brightening agent may comprise a heteroalkyl group, optionally substituted.

In some cases, the brightening agent may be an alkynyl alkoxy alkane. For example, the brightening agent may comprise a compound having the following formula,

H—C≡C—[CH₂]ₙ—O—[R¹],

wherein n is an integer between 1 and 100, and R¹ is alkyl or heteroalkyl, optionally substituted. In some cases, the R¹ is an alkyl group, optionally substituted with OH or SO₃. In some embodiments, R¹ comprises a group having the formula (R²)ₘ, wherein R² is alkyl or heteroalkyl, optionally substituted, and m is an integer between 3 and 103, such that n is less than or equal to (m−2). In some embodiments, n is an integer between 1 and 5. In some embodiments, m is an integer between 3 and 7. One specific example of a brightening agent includes, but is not limited to, propargyl-oxo-propane-2,3-dihydroxy (POPDH) and propargyl-3-sulfopropyl ether Na salt (POPS). It should be understood that other alkynyl alkoxy alkanes may also be useful as brightening agents.

In some cases, the brightening agent may comprise a hydroxy alkyne. In some embodiments, the brightening agent may comprise a compound having the following formula,

[R³]ₓ—C≡C—[R⁴]_{y},

wherein R³ and R⁴ can be the same or different and each is H, alkyl, or hydroxyalkyl, optionally substituted, and x and y can the be same or different and each is an integer between 1 and 100. In some cases, at least one of R³ or R⁴ comprises a hydroxyalkyl group. In some embodiments, x and y can be the same or different and are integers between 1-5, and at least one of R³ and R⁴ comprises a hydroxyalkyl group. In an illustrative embodiment, the hydroxy alkyne is 2-butyne-1,4-diol. It should be understood that other hydroxy alkynes may also be useful as brightening agents within the context of the invention.

In some cases, the brightening agent may be chosen from those molecules falling within the betain family, where a betain is a neutrally charged compound comprised of a positively charged cationic functional group and a negatively charged anionic functional group. Here examples of the cationic side of the betain could be ammonium, phosphonium, or pyridinium groups optionally substituted, and examples of the anionic side could be carboxylic, sulfonic, or sulfate groups. It should be understood that these functional groups are for illustration and are not intended to be limiting.

In some cases, the electrodeposition baths may include a combination of at least two brightening agents. For example, a bath may comprise both a brightening agent comprising an alkynyl alkoxy alkane and a second brightening agent comprising a hydroxy alkyne.

The baths may comprise the brightening agent in a concentration of from 0.05 g/L to 5 g/L, from 0.05 g/L to 3 g/L, from 0.05 g/L to 1 g/L, or, in some cases, from 0.01 g/L to 1 g/L. In some cases, the baths may comprise the brightening agent in a concentration of from 0.05 g/L to 1 g/L, from 0.05 g/L to 0.50 g/L, from 0.05 g/L to 0.25 g/L, or, in some cases, from 0.05 g/L to 0.15 g/L. Those of ordinary skill in the art would be able to select the concentration of brightening agent, or mixture of brightening agents, suitable for use in a particular application.

In some cases, the baths may include at least one wetting agent. A wetting agent refers to any species capable of increasing the wetting ability of the electrodeposition bath with the surface of the article to be coated. For example, the substrate may comprise a hydrophilic surface, and the wetting agent may enhance the compatibility (e.g., wettability) of the bath relative to the substrate. In some cases, the wetting agent may also reduce the number of defects within the metal coating that is produced. The wetting agent may comprise an organic species, an inorganic species, an organometallic species, or combinations thereof.

In one set of embodiments, the wetting agent may comprise an aromatic group, optionally substituted. For example, the wetting agent may comprise a naphthyl group substituted with one or more an alkyl or heteroalkyl group, optionally substituted.

In some cases, the wetting agent may comprise a sulfopropylated polyalkoxy napthol having the following formula,

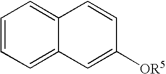

wherein R⁵ comprises an alkyl or heteroalkyl group. In some cases, R⁵ comprises a charged group, such as SO₃. For example, the wetting agent may comprise the group, —(CH₂)₃SO₃. In some embodiments, R⁵ may comprise a group having the formula (R⁶)_{q}, wherein R⁶ is alkyl or heteroalkyl, optionally substituted, and q is an integer between 1-100. In an illustrative embodiment, the wetting agent may be Ralufon NAPE 14-90 (Raschig GmbH).

In another set of embodiments, the wetting agent may comprise a fluorocarbon, optionally substituted. The fluorocarbon could be fully or partially fluorinated. The wetting agent could be chosen from the groups of anionic, non-ionic and amphoteric fluorocarbons. For example, the wetting agent may comprise a fluorocarbon substituted with a carboxylate, sulfonate, sulfate or phosphate anionic moiety.

Additives described herein can be used both individually and/or in any combinations thereof to provide improved coating quality through brightening, leveling and reduction in propensity for surface pitting.

The coating comprises one or more metals. For example, the coating may comprise an alloy (e.g., a nickel-tungsten alloy). Examples of suitable alloys may include two or more of the following elements: Ni, W, Fe, B, S, Co, Mo, Cu, Cr, Zn and Sn, amongst others. In some cases, alloys that comprises tungsten (e.g., nickel-tungsten alloys) are particularly preferred. Some specific examples of alloys include Ni—W, Ni—Fe—W, Ni—B—W, Ni—S—W, Co—W, Ni—Mo, Co—Mo and Ni—Co—W.

Various substrates may be coated to form coated articles, as described herein. In some cases, the substrate may comprise an electrically conductive material, such as a metal, metal alloy, intermetallic material, or the like. Suitable substrates include steel, copper, aluminum, brass, bronze, nickel, polymers with conductive surfaces and/or surface treatments, transparent conductive oxides, amongst others.

In general, the electrodeposition baths containing citrate can be used in connection with any electrodeposition process. Electrodeposition generally involves the deposition of a coating on a substrate by contacting the substrate with an electrodeposition bath and flowing electrical current between two electrodes through the electrodeposition bath, i.e., due to a difference in electrical potential between the two electrodes. For example, methods described herein may involve providing an anode, a cathode, an electrodeposition bath associated with (e.g., in contact with) the anode and cathode, and a power supply connected to the anode and cathode. In some cases, the power supply may be driven to generate a waveform for producing a coating. In some embodiments, at least one electrode may serve as the substrate to be coated.

The electrodeposition may be modulated by varying the potential that is applied between the electrodes (e.g., potential control or voltage control), or by varying the current or current density that is allowed to flow (e.g., current or current density control). In some embodiments, the coating may be formed (e.g., electrodeposited) using direct current (DC) plating, pulsed current plating, reverse pulse current plating, or combinations thereof. Pulses, oscillations, and/or other variations in voltage, potential, current, and/or current density, may also be incorporated during the electrodeposition process. For example, pulses of controlled voltage may be alternated with pulses of controlled current or current density. In general, during an electrodeposition process an electrical potential may exist on the substrate to be coated, and changes in applied voltage, current, or current density may result in changes to the electrical potential on the substrate. In some cases, the electrodeposition process may include the use of waveforms comprising one or more segments, wherein each segment involves a particular set of electrodeposition conditions (e.g., current density, current duration, electrodeposition bath temperature, etc.). Suitable waveforms have been described in U.S. patent application Ser. No. 12/120,568, entitled “Coated Articles and Related Methods,” filed May 14, 2008, which is incorporated herein by reference in its entirety.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method comprising:
removing a sample from an electrodeposition bath comprising a tungsten and/or molybdenum ionic species, an ionic species of a second metal, and citrate;
reacting the citrate and/or a citrate reaction product with a chemical reagent to form a product;
reacting the product with a binding agent to form a complex; and
analyzing the complex to determine the amount of citrate in the sample.

2. The method of claim 1, wherein the chemical reagent comprises a halogen.

3. The method of claim 2, wherein the halogen comprises bromine.

4. The method of claim 1, wherein the product is a halogenated product.

5. The method of claim 4, wherein the halogenated product comprises pentabromoacetone.

6. The method of claim 1, wherein the citrate and/or citrate reaction product reacts with a reagent comprising an oxidizing agent.

7. The method of claim 6, wherein the reagent further comprises sodium bromide.

8. The method of claim 6, wherein the oxidizing agent is decolorized using hydrogen peroxide after reacting the citrate and/or citrate reaction product with the reactant.

9. The method of claim 1, wherein the sample is acidified prior to reacting the citrate and/or citrate reaction product.

10. The method of claim 9, wherein the pH of the acidified sample is less than 7.

11. The method of claim 10, wherein the pH of the acidified sample is between 3 and 5.

12. The method of claim 1, wherein an organic solvent is used to extract the product from the sample.

13. The method of claim 12, wherein the organic solvent comprises a hydrocarbon.

14. The method of claim 1, wherein analysis of the complex comprises spectrophotometry.

15. The method of claim 1, wherein analysis of the complex comprises colorimetry.

16. The method of claim 1, wherein the second metal is nickel.

17. The method of claim 1, further comprising a secondary brightening agent.

18. The method of claim 1, further comprising a wetting agent.

19. The method of claim 1, wherein the binding agent comprises thiourea.

20. The method of claim 1, wherein the citrate is a complexing agent.

21. The method of claim 1, further comprising electroplating a coating on a substrate in the bath.

22. A method comprising:
removing a sample from an electrodeposition bath comprising a tungsten and/or molybdenum ionic species, an ionic species of a second metal, and citrate; and
analyzing a citrate reaction product using a spectroscopic technique to determine the amount of citrate in the sample.

23. The method of claim 22, wherein the bath includes the citrate reaction product.

24. The method of claim 22, wherein the citrate reaction product is aconitate.

25. The method of claim 22, wherein the spectroscopic technique is spectrophotometry.

26. A method comprising:
removing a sample from an electrodeposition bath comprising a tungsten and/or molybdenum ionic species, an ionic species of a second metal, and citrate and/or a citrate reaction product;
diluting the citrate and/or citrate reaction product with a diluent;
at least partially separating the citrate and/or citrate reaction product using HPLC; and
analyzing the citrate and/or citrate reaction product by mass spectrometry to determine the amount of citrate in the sample.

27. The method of claim 26, wherein the HPLC has a mobile phase composition, the mobile phase composition having an initial polarity.

28. The method of claim 27, wherein the citrate and/or citrate reaction product is diluted with a diluent, the diluent having a polarity more polar than the initial polarity of the mobile phase composition.

29. The method of claim 26, wherein the HPLC has a mobile phase composition, the mobile phase composition having an initial eluting strength.

30. The method of claim 29, wherein the citrate and/or citrate reaction product is diluted with a diluent, the diluent having an eluting strength weaker than the eluting strength of the mobile phase composition.

31. The method of claim 26, wherein the diluent comprises between about 0.1 mM and about 200 mM of a volatile buffer.

32. The method of claim 26, wherein the diluent comprises between about 10% and about 50% methanol.

33. The method of claim 26, wherein the diluent comprises between about 0% and about 1% acetonitrile.

34. The method of claim 26, wherein the diluent comprises between about 0.01% and about 2% of a volatile acid.

35. The method of claim 26, wherein the citrate and/or citrate reaction product is diluted to a concentration of less than about 100 ppm.

36. The method of claim 26, wherein the citrate and/or citrate reaction product is diluted to a concentration of between about 10 ppm and about 25 ppm.

37. The method of claim 27, wherein the mobile phase comprises ammonium acetate, formic acid, and/or methanol.

38. The method of claim 26, wherein the citrate reaction product is aconitate.
